(19) United States
(12) Patent Application Publication
(10) Pub. No.: US 2025/0002442 A1
(43) Pub. Date: Jan. 2, 2025

(54) SYNTHESIS OF INTERMEDIATES FOR PRODUCING PROSTACYCLIN DERIVATIVES

(71) Applicant: United Therapeutics Corporation, Silver Spring, MD (US)

(72) Inventors: Hitesh Batra, Herndon, VA (US); Sudersan M. Tuladhar, Silver Spring, MD (US); David A. Walsh, Spotsylvania, VA (US)

(73) Assignee: United Therapeutics Corporation, Silver Spring, MD (US)

(21) Appl. No.: 18/886,228

(22) Filed: Sep. 16, 2024

Related U.S. Application Data
(63) Continuation of application No. 17/577,222, filed on Jan. 17, 2022.
Application No. 17/577,222 is a division of application No. 16/991,460, filed on Aug. 12, 2020, now Pat. No. 11,225,452.
Application No. 16/991,460 is a continuation of application No. 16/264,053, filed on Jan. 31, 2019, now Pat. No. 10,774,027.
Application No. 16/264,053 is a continuation of application No. 15/439,189, filed on Feb. 22, 2017, now Pat. No. 10,196,342.
Application No. 15/439,189 is a continuation of application No. 14/887,298, filed on Oct. 19, 2015, now Pat. No. 9,593,061.
(60) Provisional application No. 62/066,009, filed on Oct. 20, 2014.

(51) Int. Cl. C07C 67/343; C07C 45/64; C07C 45/67; C07C 45/78; C07C 45/81; C07C 67/31; C07C 67/333

(52) U.S. Cl. CPC C07C 67/343; C07C 45/64; C07C 45/67; C07C 45/78; C07C 45/81; C07C 67/31; C07C 67/333; C07C 2603/14

(57) ABSTRACT

The present disclosure provides regioselective methods for synthesizing intermediates useful in making prostacyclin derivatives, such as treprostinil.

### RELATED APPLICATIONS

The present application is a Continuation of U.S. application Ser. No. 17/577,222, filed Jan. 17, 2022, which is a divisional of U.S. application Ser. No. 16/991,460, filed Aug. 12, 2020, which is a Continuation of U.S. application Ser. No. 16/264,053, filed Jan. 31, 2019, which is a Continuation of U.S. application Ser. No. 15/439,189, filed Feb. 22, 2017, which is a Continuation of U.S. application Ser. No. 14/887,298, filed Oct. 19, 2015, which claims priority to U.S. provisional application No. 62/066,009 filed Oct. 20, 2014, which is incorporated herein by reference in its entirety.

### FIELD

The present application generally relates to chemical synthetic methods and in particular, to synthesis of aldehyde compounds, which may be useful in preparation of pharmaceutically active prostacyclins, such as treprostinil.

### SUMMARY

A method of producing a compound of formula 3:

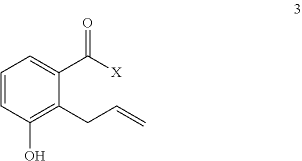

comprising heating a solution comprising a compound of formula 2:

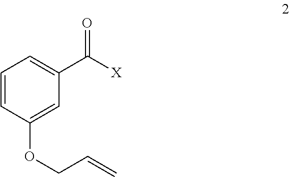

and an organic solvent, and wherein X is hydrogen, an alkoxy group or OR², wherein R² is unsubstituted or substituted aryl, or unsubstituted or substituted benzyl. The heating can comprise irradiating the solution with microwave radiation.

### DETAILED DESCRIPTION

Unless otherwise specified, “a” or “an” means “one or more.”

The term “aryl,” alone or in combination with another radical, means a carbocyclic aromatic system containing one, two, or three rings wherein such rings may be attached together in a pendent manner or may be fused. The term “aryl” embraces aromatic radicals including, but not limited to, phenyl, naphthyl, tetrahydronaphthyl, indyl, and biphenyl. A substituted aryl group may be optionally substituted at one or more positions with one or more substituents, which may be independently selected from the group consisting of —NO₂, —CN, halogen (e.g., —F, —Cl, —Br or —I), (C₁-C₃) alkyl, halo (C₁-C₃) alkyl, (C₁-C₃) alkoxy, and halo (C₁-C₃) alkoxy.

Prostacyclin derivatives are useful pharmaceutical compounds possessing activities, such as platelet aggregation inhibition, gastric secretion reduction, lesion inhibition, and bronchodilation.

Treprostinil, the active ingredient in Remodulin®, Tyvaso®, and Orenitram™ was first described in U.S. Pat. No. 4,306,075. Methods of making treprostinil and other prostacyclin derivatives are described, for example, in Moriarty, et al., J. Org. Chem. 2004, 69, 1890-1902, Drug of the Future, 2001, 26(4), 364-374, U.S. Pat. Nos. 6,441,245, 6,528,688, 6,700,025, 6,809,223, 6,756,117, 8,461,393, 8,481,782; 8,242,305, 8,497,393, and 8,940,930; U.S. Published Patent Application Nos. 2012-0197041, 2013-0211145, 2014-0024856, 2015-0025255; and PCT Publication No. WO2012/009816.

Various uses and/or various forms of treprostinil are disclosed, for examples, in U.S. Pat. Nos. 5,153,222, 5,234,953, 6,521,212, 6,756,033, 6,803,386, 7,199,157, 6,054,486, 7,417,070, 7,384,978, 7,879,909, 8,563,614, 8,252,839, 8,536,363, 8,410,169, 8,232,316, 8,609,728, 8,350,079, 8,349,892, 7,999,007, 8,658,694, 8,653,137, 9,029,607, 8,765,813, 9,050,311, U.S. Published Patent Application Nos. 2009-0036465, 2008-0200449, 2010-0076083, 2012-0216801, 2008-0280986, 2009-0124697, 2014-0275616, 2014-0275262, 2013-0184295, 2014-0323567, PCT Publication No. WO00/57701.

Treprostinil, also known as UT-15, LRX-15, 15AU81, UNIPROST™, BW A15AU; and U-62,840 has the following chemical formula:

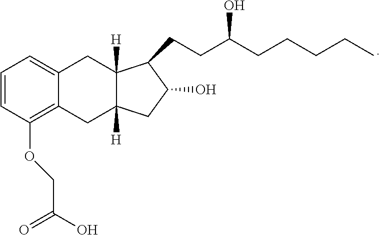 [SMILES: CCCCC[C@H](O)CC[C@@H]1[C@H]2Cc3cccc(OCC(=O)O)c3C[C@H]2C[C@H]1O]

The present inventors developed novel methods for synthesizing aldehyde compounds. These aldehyde compounds can be intermediates in processes for producing treprostinil and other prostacyclin derivatives or pharmaceutically acceptable salts or esters thereof, such as the processes disclosed in Moriarty, et al. J. Org. Chem. 2004, 69, 1890-1902, Drug of the Future, 2001, 26(4), 364-374, U.S. Pat. Nos. 6,441,245, 6,528,688, 6,700,025, 6,809,223, 6,756,117, 7,417,070, 8,461,393, 8,481,782, 8,242,305, and 8,497,393, U.S. Published Patent Application Nos. 2012-0190888 and 2012-0197041, PCT Publication No. WO2012/009816.

In one embodiment, the present disclosure provides a method of producing a compound of formula 3:

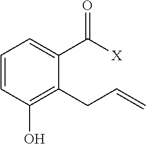

by heating a solution comprising a compound of formula 2

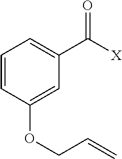

and an organic solvent. In some embodiments, the heating comprises irradiating said solution with a microwave radiation. In the above formulae, X may be hydrogen, an alkoxy group or OR₂, where unsubstituted or substituted aryl, or unsubstituted or substituted benzyl.

Use of microwave radiation in chemistry is known to those skilled in the art. See e.g. Polshettiwar, V.; et al Accounts of Chemical Research 2008, 41 (5), 629-639; Bowman, M. D.; Organic Process Research & Development 2007, 12 (1), 41-57; Sauks, J. M. et al., Organic Process Research & Development 2014, 18 (11): 1310-1314; Microwaves in organic synthesis, Andre Loupy (ed), Wiley-VCH, Weinheim, 2006; Microwaves in organic synthesis. Thermal and non-thermal microwave effects, Antonio de la Hoz, Angel Diaz-Ortiz, Andres Moreno, Chem. Soc. Rev., 2005, 164-178; Developments in Microwave-assisted Organic Chemistry. C. Strauss, R. Trainor. Aust. J. Chem., 48 1665 (1995); Microwaves in Organic and Medicinal Chemistry, 2nd, Completely Revised and Enlarged Edition, Wiley-VCH, Weinheim, 2012.

In certain embodiments, the heating with the microwave radiation may be performed at a temperature ranging from 150° C. to 200° C. or from 175° C. to 195° C., such as within a range of 182-185° C.

The heating with the microwave radiation may performed for 1 hour to 30 hours, from 2 hours to 25 hours, from 2 hours to 20 hours, from 2 hours to 15 hours, from 3 hours to 14 hours, any value or any subrange within these ranges. In some embodiments, the reaction times may be significantly lower compared to the ones of prior art methods.

Use of microwave radiation or conventional heating to heat a solution comprising the compound of formula 2 may result in producing the isomer of the compound of formula 3, a compound of formula 4:

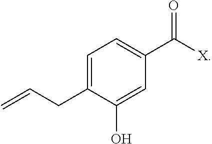

Selection of appropriate solvents can result in separating the compound of formula 3 from the compound of formula 4 through a selective crystallization of one of the isomers. Appropriate solvents include, but are not limited to, 1,2-dichlorobenzene or tetrahydronaphtalene. This means that one of the isomers, e.g. the compound of formula 3 may crystallize, while the other of the isomers, e.g. the compound of formula 3, would remain dissolved in the solvent. In some embodiments, the methods described herein allow producing a high-purity batch of the compound of formula 3 having a purity of at least 95% by weight of the composition, at least 96% or at least 97%, at least 98%, at least 98.5%, at least 99%, at least 99.1%, at least 99.2%, at least 99.3%, at least 99.4%, at least 99.5%, at least 99.6%, least 99.7%, at least 99.8%, or at least 99.9% by weight of the composition.

Selective crystallization allows for production high-purity batches of the compound of formula 3 without performing column chromatography purifications, which may save manpower, large volumes of solvents, and lost product.

In some embodiments, the methods described herein permit production of the compound of formula 3 in large quantities, such as at least 10 g, at least 20 g, at least 30 g, at least 50 g, at least 80 g, at least 100 g, at least 150 g, at least 200 g, at least 250 g, at least 300 g, at least 400 g, at least 500 g, at least 800 g, at least 1000 g, at least 1200 g, at least 1500 g, at least 2000 g, at least 2500 g, at least 3000 g, at least 3500 g, at least 4000 g, at least 4500 g, at least 5000 g, at least 6000 g; at least 7000 g, at least 8000 g, at least 9000 g, or at least 10000 g.

In some embodiments, X may be hydrogen or an alkoxy group, which may be, for example, C₁-C₈ alkoxy group or C₁-C₄ alkoxy group, such as methoxy or ethoxy. The solvent in the solution comprising the compound of formula 2 may comprise, for example, at least one of triglyme, N-methylpyrrolidinone, tetradecane, tetrahydronaphthalene, Dowtherm A™ p-chlorophenol, 1,2-dichlorobenzene, and diphenyl ether. In some embodiments, the solvent in the solution comprising the compound of formula 2 may comprise, for example, at least one of 1,2-dichlorobenzene and tetrahydronaphtalene.

In some embodiments, X may be OR₂, where R₂ is C₁₋₄ alkyl, unsubstituted or substituted aryl, or unsubstituted or substituted benzyl. The C₁₋₄ alkyl can be methyl, ethyl, propyl, isopropyl, n-butyl, isobutyl, sec-butyl or tert-butyl. Non-limiting examples of X include OCH₃; OCH₂CH₃ and OCH₂Ph. The solvent in the solution comprising the compound of formula 2 may comprise, for example, at least one of triglyme, N-methylpyrrolidinone, tetradecane, tetrahydronaphthalene, Dowtherm A™ (a mixture of 26.5% diphenyl and 73.5% diphenyl oxide), p-chlorophenol, 1,2-dichlorobenzene and diphenyl ether. In some embodiments, the solvent in the solution comprising the compound of formula 2 may comprise, for example, at least one of 1,2-dichlorobenzene and tetrahydronaphtalene.

In some embodiments, the compound of formula 3 may be converted to a compound of formula 5

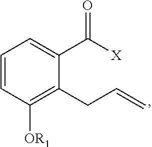

using O-alkylation, wherein R₁ is selected from (a) benzyl or substituted benzyl and (b) CH₂COOR₄, wherein R₄ is C₁₋₄ alkyl, such as methyl, ethyl, propyl, isopropyl, n-butyl, isobutyl, sec-butyl or tert-butyl. The purity of a batch of the compound of formula 5 may be as high as purity of the batch of the compound of formula 3. O-alkylation of phenol is known in the art, see e.g. P. G. M. Wuts and T. W. Greene, “Greene's Protecting Groups in Organic Synthesis”, John Wiley & Sons, Inc. 2007, 4th edition; page 390; F. Martin and P. J. Garrison; J. Org. Chem., 1982, 47, 1513; T. Satoh, M. Ikeda, M. Miura and M. Nomura: J. Org. Chem., 1997, 62, 4877.

In some embodiments, when R₁ is hydrogen, O-alkylation may be performed by reacting the compound of formula 3 with benzyl halides, such as BnCl, BnBr or BnI. Such reaction may be performed in an alkaline solution, which may be, for example, an aqueous solution of K₂CO₃. Other O-alkylation conditions of phenol are known in the art, see e.g. P. G. M. Wuts and T. W. Greene, “Greene's Protecting Groups in Organic Synthesis,” John Wiley & Sons, Inc., 2007, 4th Edition, page 370.

A substituted benzyl group may be optionally substituted at one or more meta, ortho, or para positions with one or more substituents, which may be independently selected from the group consisting of —NO₂, —CN, halogen (e.g., —F, —Cl, —Br or —I), (C₁-C₃) alkyl, halo (C₁-C₃) alkyl, (C₁-C₃) alkoxy, and halo (C₁-C₃) alkoxy.

In some embodiments, the compound of formula 5 may then converted to treprostinil or its pharmaceutically acceptable salt through a process comprising Pauson-Khand cyclization. Such processes are disclosed, for example, in U.S. Pat. Nos. 8,481,782, 6,700,025, 6,809,223, 6,441,245, 6,765,117, 6,528,688, and U.S. Published Patent Application Nos. 2012-0190888 and 2012-0197041.

In some embodiments, when X is OR₂, where R₂ is C₁₋₄ alkyl, the compound of formula 3 may be used for forming a compound of formula 11

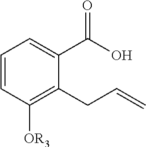

through O-alkylation and hydrolysis, wherein R₃ is C₁₋₄ alkyl or a phenolic protecting group. O-alkylation is known in the art, see e.g. P. G. M. Wuts and T. W. Greene, “Greene's Protecting Groups in Organic Synthesis”, John Wiley & Sons, Inc. 2007, 4th edition; page 390; F. Martin and P. J. Garrison; J. Org. Chem., 1982, 47, 1513; T. Satoh, M. Ikeda, M. Miura and M. Nomura: J. Org. Chem., 1997, 62, 4877. Hydrolysis of esters is known in the art as well. In some embodiments, the selective hydrolysis may be performed using a bulky base, such as barium hydroxide, cesium hydroxide, or trialkyl ammonium hydroxide. In some embodiments, the trialkyl ammonium hydroxide can be tributyl ammonium hydroxide or trimethyl ammonium hydroxide. In some embodiments, a base for selective hydrolysis may be an alkali metal hydroxide. Although the use of bulky bases, such as barium hydroxide and cesium hydroxide, other alkali metal hydroxides, such as potasium hydroxide and sodium hydroxide may be used if they can provide selective hydrolysis of one of regioisomers. A base used in selective hydrolysis may selectively hydrolyze one (less hindered) isomer, and this may provide the advantage of separating the desired isomer in the present synthesis, see e.g. Scheme 2. Ester hydrolysis using various conditions are disclosed, for example, in P. G. M. Wuts and T. W. Greene, “Greene's Protecting Groups in Organic Synthesis”, John Wiley & Sons, Inc. 2007, 4th edition; page 543-544.

As used herein, “a phenolic protecting group” is a modification that protects the hydroxyl group from participating in reactions that are occurring in other parts of the molecule. Suitable phenolic protecting groups are well known to those of ordinary skill in the art and include those found in P. G. M. Wuts and T. W. Greene, “Greene's Protecting Groups in Organic Synthesis”, John Wiley & Sons, Inc. 2007, 4th edition; page 367-430, the entire teachings of which are incorporated herein by reference. Exemplary phenolic protecting groups include, but are not limited to, actetyl, benzoyl, benzyl, p-methoxyethoxymethyl, methoxymethyl, dimethoxytrityl, p-methoxybenzyl, trityl, silyl (e.g., trimethylsilyl (TMS), tert-butyldimethylsilyl (TBMDS), tert-butyldimethylsilyloxymethyl (TOM) or triisopropylsilyl (TIPS), tetrahydropyranyl (THP), methyl and ethoxyethyl (EE).

In some embodiments, the compound of formula 11 may then converted to treprostinil or its pharmaceutically acceptable salt through a process comprising Pauson-Khand cyclization. Such processes are disclosed, for example, in U.S. Pat. Nos. 8,481,782, 6,700,025, 6,809,223, 6,441,245, 6,765,117, and 6,528,688 and U.S. Published Patent Application Nos. 2012-0190888 and 2012-0197041.

In some embodiments, the compound of formula 2 used for making the compound of formula 3 may be produced by allylating a compound of formula 1

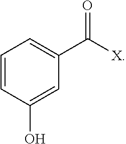

Allylation reactions are disclosed, for example, in By Nicolaou, K. C. et. al.; From Chemistry—A European Journal, 7(17), 3798-3823; 2001; Moriarty R. M. et. al.; From PCT Int. Appl., 2002053517, 11 Jul. 2002; Mmutlane, Edwin M. et. al. From Organic & Biomolecular Chemistry, 2(17), 2461-2470; 2004; Paul, Caroline E. et. al.; From Chemical Communications (Cambridge, United Kingdom), 48(27), 3303-3305; 2012. In some embodiments, the disclosed methods may provide one or more of the following advantages: a) reduce reaction times; b) provide high purity batches of a desired isomer by using selective crystallization of the desired isomer depending on the solvents used; c) eliminate column chromatographic purifications and thereby, significantly save manpower and large volume of solvents; d) be scaled up to kilo-gram quantities; e) the compound of formula 3 may be used to synthesize various O-ethers, esters and acid functionalities, which may be useful synthons for the synthesis of prostacyclins, such as treprostinil. Embodiments described herein are further illustrated by, though in no way limited to, the following examples.

### EXAMPLES

A protocol (Scheme 1) has been developed for the synthesis of 2-allyl-3-hydroxy benzaldehyde (3) via Claisen rearrangement of allyl-ether of 3-hydroxybenzaldehyde (2) using microwave. The allyl ether (2) is heated by irradiating microwaves in various solvents (see Table 1) app. at 180° C. for 7-12 hrs. The use of microwave enhances the rate of reaction and significantly may reduce the reaction times over conventional thermal rearrangement. Also, the desired isomer (3) crashes out as white to off-white solid leaving the non-desired aldehyde (regio-isomer) (4) in mother liquor.

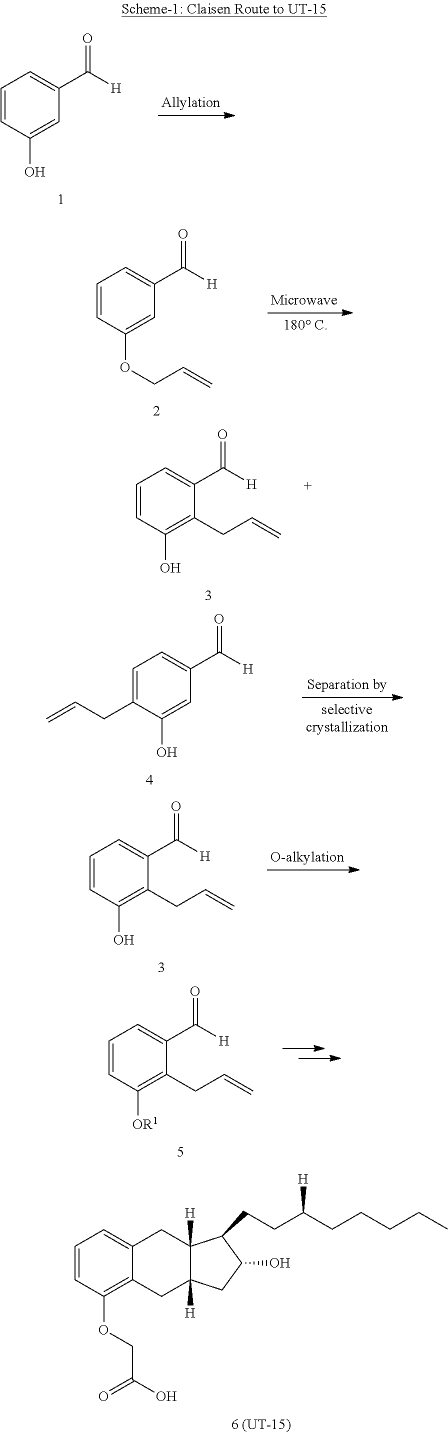

Another methodology developed (Scheme 2) involves the use of methyl 2-allyloxybenzoate (8) for obtaining the Claisen rearranged product methyl 2-allyl-3-hydroxybenzoate (9). During the rearrangement mixture of regio-isomers are produced in app. 3:1 or 2:1 ratio and separation can be challenging. As one can visualize, ester 9 is sterically hindered as compared to ester 10 due to the presence of allyl group at the ortho position and hence is less susceptible to attack by a base for hydrolysis. The inventors took advantage of this steric hinderance available in the molecule and selectively hydrolyzed the regio-isomer 10 to acid and this was subsequently separated by acid-base work-up to obtain pure 2-allyl-3-hydroxybenzoate (9). This is an important intermediate as this can be used to obtain aldehyde (5) by reducing with various chemical reagents such as lithium aluminum hydride (LAH), diisobutyl lithium aluminum hydride (DIBAL) etc.

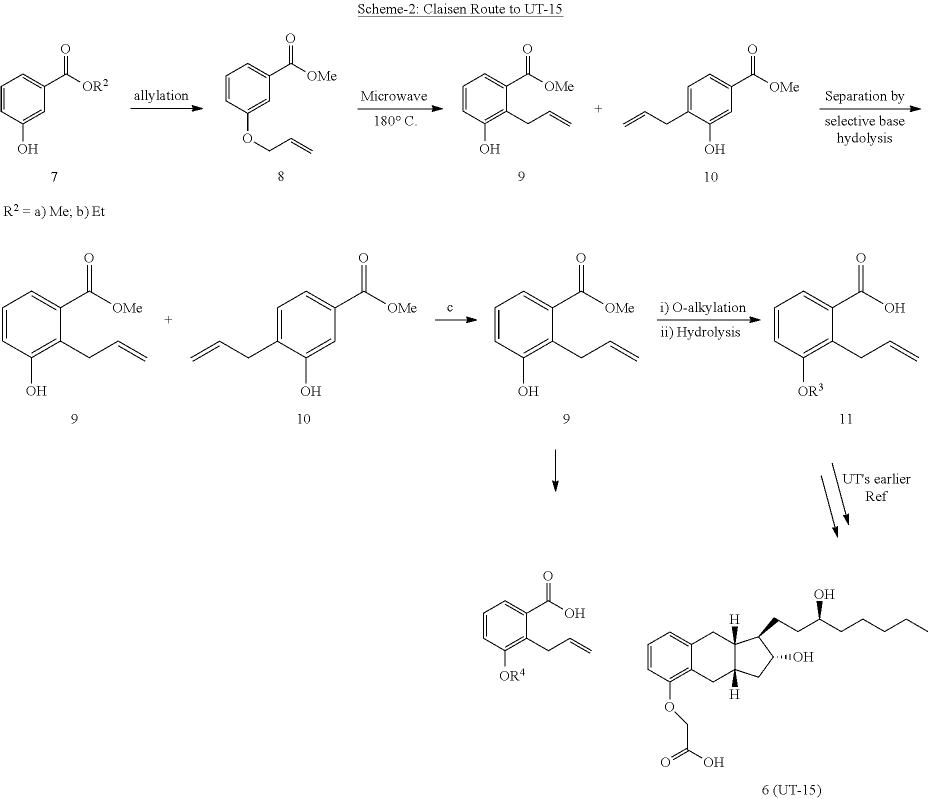

A brief overview of the experiments carried on Claisen rearrangement is given below in table 1.

TABLE 1
Study Results on Claisen Rearrangement (2→3)

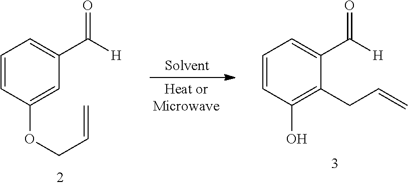

| S.No. | Lot # | Solvent | Amount | Temp. | Time |
|---|---|---|---|---|---|
| 1 | D- | Triglyme | 1.0 g | 180-182° | 22 h |
| | 1057- | | | C. | |
| | 087 | | | | |
| 2 | D- | N-methyl | 1.0 g | 180-182° | 22 h |
| | 1057- | pyrrolidinone | | C. | |
| | 088 | | | | |
| 3 | D- | Carbitol | 1.0 g | 180-182° | 19 h |
| | 1057- | | | C. | |
| | 089 | | | | |
| 4 | D- | Tetradecane | 1.0 g | 190-192° | 19 h |
| | 1057- | | | C. | |
| | 090 | | | | |
| 5 | D- | Tetrahydro- | 3.0 g | 180-182° | 11 h |
| | 1057- | naphthalene | | C. | |
| | 063 | | | | |
| 6 | D- | Dowtherm A ™ | 1.0 g | 190-192° | 6 h |
| | 1057- | (mixture of | | C. | |
| | 066 | diphenyl 26.5% | | | |
| | | and diphenyl | | | |
| | | oxide 73.5%) | | | |
| 7 | D- | Neat | 4.0 | 180-182° | 30 |
| | 1057- | | 15.0 g | C. | min. |
| | 064 | | | | |
| | D- | | | | |
| | 1057- | | | | |
| | 069 | | | | |
| 8 | D- | Para- | 1.0 g | 160-162° | 1 h |
| | 1057- | cholorophenol | | C. | |
| | 144 | | | | |
| 9 | D- | N-methyl | 1.0 g | 180-182° | 1 h |
| | 1057- | pyrrolidinone | | C. | |
| | 145 | | | | |
| 10 | D- | Tetrahydro | 1.0 g | 182-185° | 11 h |
| | 1057- | naphthalene | | C. | |
| | 147 | | | | |
| 11 | D- | 1,2- | 1.0 g | 175-178° | 24 h |
| | 1057- | Dichlorobenzene | | C. | |
| | 148 | | | | |
| 12 | D- | 1,2- | 5 g | 182-185° | 7 h |
| | 1057- | Dichlorobenzene | | C. | |
| | 153 | | | Microwave | |
| 13 | D- | Tetrahydro- | 50 g | 182-185° | 4 h |
| | 1057- | naphthalene | | C. | |
| | 155 | | | Microwave | |
| 14 | D- | Tetrahydro- | 308 g | 182-185° | ~7 h |
| | 1057- | naphthalene | | C. | |
| | 172 | | | Microwave | |

### General Experimental

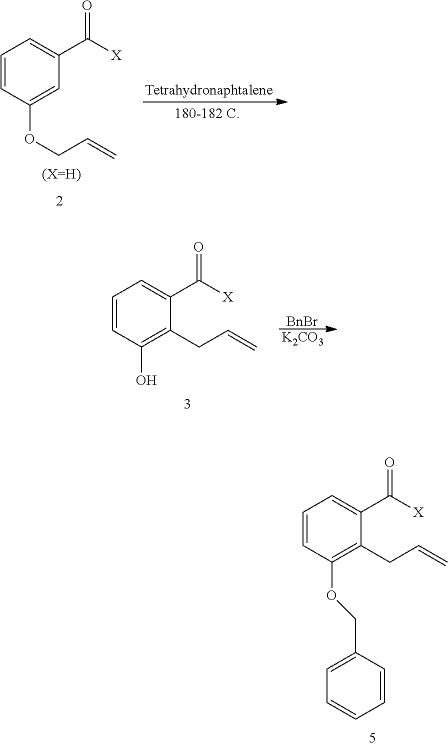

### Synthesis of 3-Hydroxy-2-allylbenzaldehyde (8)

| Name | MW | Amount | Mole |
|---|---|---|---|
| Allyl ether (7) | NA | 308 g | NA |
| Tetrahydronaphthalene | NA | 300 mL | NA |

To a 3000 ml one neck, round bottom flask equipped with a condenser and thermometer was added allylether (7) (308 g) and tetrahydronaphthalene (300 mL). This reaction mixture was heated slowly up to 180-182° C. (ramped the temp. in 5-10 minutes, internal temperature) in a microwave (power: 1500 Watts) and was kept at this temperature while stirring for 7-8 h. At this stage the reaction mixture turned brown and the reaction mixture was cooled to room temperature followed by cooling at 0 to 5° C. for 30 minutes. The solid was filtered and dried to obtain off-white solid (3-hydroxy-2-allylbenzaldehyde, 8) 145.5 g (47%). The compound (8) was characterized by spectral data. Completion of reaction was monitored by TLC using a thin layer silica gel plate; eluent: 15% ethyl acetate in hexanes.

### Synthesis of 2-Allyl-3-benzyloxybenzaldehyde (1)

A 500-mL round-bottom flask equipped with a magnetic stirrer and stir bar was charged with a solution of 3-hydroxy-2-allyl benzaldehyde (8) (25 g in 250 mL acetone), benzyl bromide (28.36 g, 1.05 eq.) and potassium carbonate (54.4 g, 2.5 eq.). The mixture was stirred at room temperature overnight (progress of reaction was monitored by TLC). The suspension was filtered and the filtrate was evaporated in vacuo to afford a crude semi-solid mass. This was taken in 550 ml of hexanes and stirred for 2h. The solid was crashed out of hexanes and filtered to obtain 2-allyl-3-benzyloxybenzaldehyde (1), yield 36.6 g (95%). The compound was confirmed by spectral data. Completion of reaction was monitored by TLC using a thin layer silica gel plate; eluent: 20% ethyl acetate in hexanes.

Although the foregoing refers to particular preferred embodiments, it will be understood that the present invention is not so limited. It will occur to those of ordinary skill in the art that various modifications may be made to the disclosed embodiments and that such modifications are intended to be within the scope of the present invention. All of the publications, patent applications and patents cited in this specification are incorporated herein by reference in their entirety.

What is claimed is:

1. In a process for producing treprostinil from an intermediate compound of formula 3, the improvement comprising adding a solvent to a mixture of compounds of formula 3 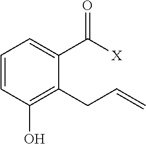
and formula 4 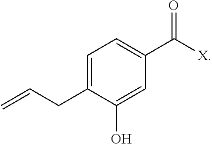
wherein the solvent selectively crystallizes the compound of formula 3, leaving the compound of formula 4 dissolved in the solvent, wherein X is hydrogen, alkoxy, OR², unsubstituted or substituted aryl, or unsubstituted or substituted benzyl, and
wherein R² is C₁₋₄ alkyl, unsubstituted or substituted aryl, or unsubstituted or substituted benzyl.

2. The process of claim 1, wherein the solvent is 1,2-dichlorobenzene.

3. The process of claim 1, wherein the solvent is tetrahydronaphthalene.

4. The process of claim 1, wherein the mixture is obtained by performing a Claisen rearrangement of a compound of formula (2) 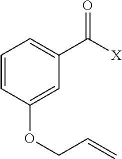
in the solvent.

5. The process of claim 4, wherein the Claisen rearrangement comprises irradiating the compound of formula (2) with a microwave radiation.

6. The process of claim 5, wherein said irradiating is performed at a temperature ranging from 180° C. to 182° C.

7. The method of claim 1, wherein X is OR^{2.}

8. The method of claim 7, wherein R² is methyl.

9. The method of claim 7, wherein R² is ethyl.

10. The method of claim 1, further comprising isolating the compound of formula 3.

11. The method of claim 10, further comprising converting the compound of formula 3 to treprostinil.
